(19) United States
(12) Patent Application Publication
(10) Pub. No.: US 2024/0379245 A1
(43) Pub. Date: Nov. 14, 2024

(54) A METHOD AND A SYSTEM FOR PROVIDING DATA FOR DIAGNOSIS AND PREDICTION OF AN OUTCOME OF CONDITIONS AND THERAPIES

(71) Applicant: Inteneural Networks Inc., Chicago, IL (US)

(72) Inventors: Kris B. SIEMIONOW, Chicago, IL (US); Paul LEWICKI, Tulsa, OK (US)

(21) Appl. No.: 18/574,377

(22) PCT Filed: Jun. 27, 2022
(86) PCT No.: PCT/US2022/035062

§ 371 Date: Dec. 27, 2023

Related U.S. Application Data
(60) Provisional application No. 63/215,491, filed on Jun. 27, 2021.

(51) Int. Cl. G16H 50/70; G16H 10/60; G16H 30/20; G16H 30/40

(52) U.S. Cl. CPC G16H 50/70; G16H 10/60; G16H 30/20; G16H 30/40

(57) ABSTRACT

A method that includes: receiving a medical image of an examined patient, the medical image covering an area or volume of the examined patient's anatomy; inputting the medical image to a classifying neural network to generate descriptors; receiving additional data of the examined patient; providing an other patients history database comprising other patients' records, the records including the descriptors, the additional data and a clinical outcome of individual patients; determining a patient from the other patient's history database being a closest match to the examined patients in terms of features of the descriptors to be a digital twin patient; presenting the clinical outcome of the digital twin patient.

### TECHNICAL FIELD

The invention relates to a method and a system for providing data for diagnosis and prediction of an outcome of a variety of conditions and therapies using historical data collected from in a database using multi-criterial search among characteristic features, including medical images.

### BACKGROUND

In general, diagnosing conditions and predicting an outcome of treatments and therapies is a difficult task, because of a multitude of contributing factors. The process involves in many cases gathering examinations and information from multiple sources to arrive at a final decision. Availability of historical patient data is one of the most valuable tools to assist in the decision making. A variety of features associated with a wide range of therapies and conditions are collected constantly as medical records.

### SUMMARY OF THE INVENTION

Finding cases that are similar according to a set criteria might help with a more accurate diagnosis or selecting a therapy with the best possible outcome based on historical data. However, searching large datasets is not an easy task. While the similarity of some categorical or numerical features (age, sex) can be easily checked, finding similar more complex diagnostic results such as blood test or in particular, medical images such as computed tomography, x-ray, magnetic resonance imaging or ultrasound imaging can be a challenging task.

In one aspect, the invention relates to a method comprising receiving a medical image of an examined patient, the medical image covering an area or volume of the examined patient's anatomy; inputting the medical image to a classifying neural network to generate descriptors; receiving additional data of the examined patient; providing an other patients history database comprising other patients' records, the records including the descriptors, the additional data and a clinical outcome of individual patients; determining a patient from the other patient's history database being a closest match to the examined patients in terms of features of the descriptors to be a digital twin patient; and presenting the clinical outcome of the digital twin patient.

The classifying neural network can be an ImageNet.

The method may comprise determining the digital twin patient by finding a set of most similar candidates from the patient history database using a first technique and next finding the digital twin patient from the set of the most similar candidates using a second technique.

In another aspect, the invention relates to a computer-implemented system, comprising at least one nontransitory processor-readable storage medium that stores at least one of processor-executable instructions or data; and at least one processor communicably coupled to at least one nontransitory processor readable storage medium, wherein at least one processor is configured to perform the steps of the method as described herein.

The invention presents a methodology for searching for the most similar cases in vast databases of medical history, involving all related data, including medical imaging. The result of the search is a subset of similar cases with complete history and treatment outcomes—the so-called digital twins.

### BRIEF DESCRIPTION OF DRAWINGS

Various embodiments are herein described, by way of example only, with reference to the accompanying drawings, wherein:

FIG. 1 shows a flowchart of a method for diagnosing and predicting an outcome of a disease or therapy in accordance with an embodiment;

FIG. 2 shows a structure of a system for diagnosing and predicting an outcome of a disease or therapy in accordance with an embodiment;

FIGS. 3A, 3B, 3C, 3D, 3E, 3F show examples of input medical images;

FIG. 4 shows a structure of a classifying CNN;

FIG. 5 shows a siamese architecture of two CNNs;

FIG. 6 shows a flowchart of a method for searching for a digital twin;

FIG. 7A shows an example of a digital profile being examined;

FIGS. 7B, 7C, 7D show examples of profiles of most similar candidates;

FIG. 8A shows a profile being examined overlaid on a digital twin profile;

FIG. 8B shows a development of the profile being examined over time;

FIG. 9 is a schematic that shows components of a computer-implemented system in accordance with an embodiment of the invention.

### DETAILED DESCRIPTION

The following detailed description is of the best currently contemplated modes of carrying out the invention. The description is not to be taken in a limiting sense but is made merely for the purpose of illustrating the general principles of the invention, since the scope of the invention is best defined by the appended claims.

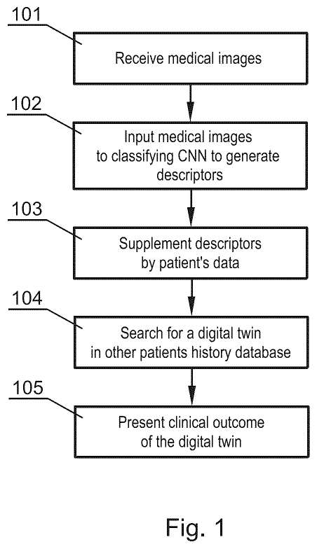

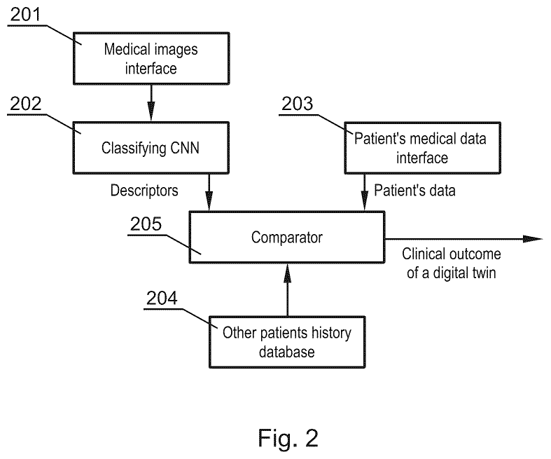

FIG. 1 shows a flowchart of a method for diagnosing and predicting an outcome of a disease or therapy in accordance with an embodiment of the invention and FIG. 2 shows a corresponding system.

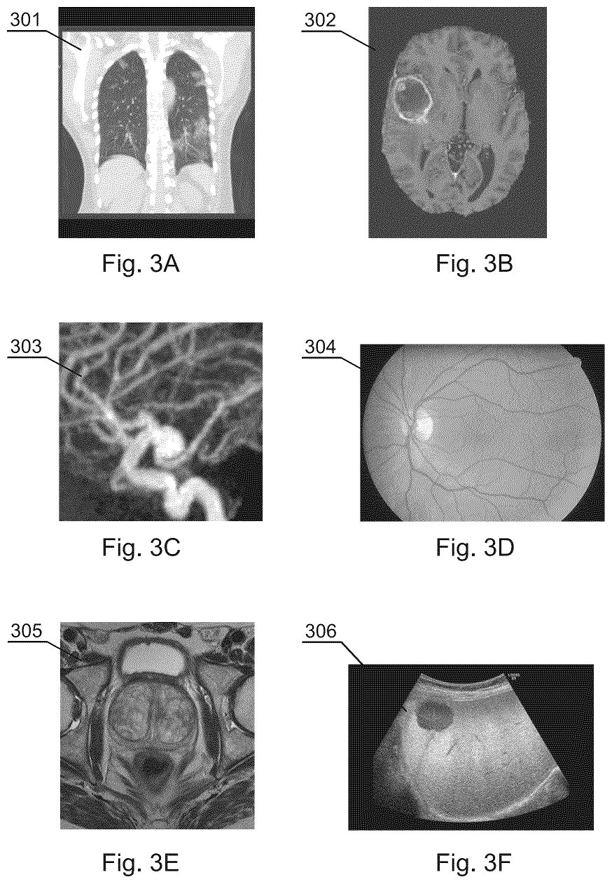

The method starts in step 101 with receiving medical images, such as a 2D or 3D medical images sourced from magnetic resonance (MR) time-of-flight (TOF), X-ray, ultrasonography or CT angiography scan data including blood vessel information. These images shall cover the area or volume that is adapted to the needs of the application, as some of the diagnostic procedures operate on the whole acquired image of volume, and others on the specific area or volume of interest. The images are received via the image interface 201, for example an external system that collects images from a MR or CT scanner and performs their preprocessing. FIGS. 3A-3F show example input medical images, including:
FIG. 3A—an image 301 of a 2D chest CT
FIG. 3B—an image 302 of a brain 3D MRI (tumor visible)
FIG. 3C—an image 303 of a maximum intensity projection (MIP) region of interest 2D image from a 3D magnetic resonance angiography depicting an aneurysm
FIG. 3D—an image 304 of a fundus image
FIG. 3E—an image 305 of a prostate MRI slice
FIG. 3F—an image 306 of a 2D liver ultrasound with tumor (top left)
   Next, in step 102, the input medical images are input to a classifying neural network 202 generating descriptors. The descriptors can have the form of real-valued or binary embedding vectors. The neural network performs the task of generating the vector of values uniquely describing the image or volume directly from the raw image or volume data. Aside from the embedding vectors, the images might be used to generate other case-specific descriptors. For example, a medical image in a form of a chest CT might be used to compute a histogram representing the number and the size of the lung nodules. In the case of an aneurysm, the vessels and the lesion profiles according to their size, shape and morphology captured for example as image or volume moments can be computed. (i.e., its structure defined as a 3D model) and provided as descriptors.

In step 103, the descriptors are supplemented by additional data of the patient (collected from patient's data interface 203, such as a medical information system) on patient's concomitant diseases and condition affecting the dynamics of the aneurysm growth, such as (but not limited to) connective-tissue disorders, hypertension, hypercholesterolemia, smoking history and family incidence of subarachnoid haemorrhage.

In step 104, an other patients' history database 204 is searched by means of a comparator 205 to establish presence of a so-called digital twin, i.e., a nearly identical case in terms of image descriptors and additional data. The database includes data in a format corresponding to the descriptors and additional data of the patient output in steps 102 and 103, so that the data can be easily searched for. The data of patients in the database further includes the known clinical outcome of a particular patient.

In step 105, the result of the search is presented, including information about the clinical outcome of the digital twin or twins that have been found, which can assist physicians in predicting patients' disease progression (i.e., risk stratification based on digital profile similarity). For example, the digital twin aneurysm shape 801 can be overlaid on the examined shape 802 of lesion so that differences can be easily identified, as shown in FIG. 8A. Further, the development of aneurysm can be easily tracked by overlaying successive scans 803, 804 of the same regions, as shown in FIG. 8B.

The vascular pathology database 204 comprises raw and processed (for example segmented) medical images of anatomy collected using a range of modalities (CT, MR, ultrasound, . . . ), depending on the needs of the procedure and corresponding patients' data. Longitudinal tracking of the patients in the database can be performed in real time and the database can be updated accordingly should the outcome change. The database therefore consists of information related to disease progression for a particular patient over time. The medical images are characterized and labeled in terms of their specific characteristics and properties, such as size, shape, geometry, architecture, completeness, and morphology. If any kind of pathology (e.g., aneurysm or arterio-venous malformation) is associated with a particular patient's medical image, it is also characterized as above. This creates a database consisting of multiple patient digital profiles with a known medical outcome. This database 204 is then used to compare individual medical images and other associated data of a patient being examined against those in the database in order to help predict disease progression, plan further diagnostic steps and stratify the patient's risk profile.

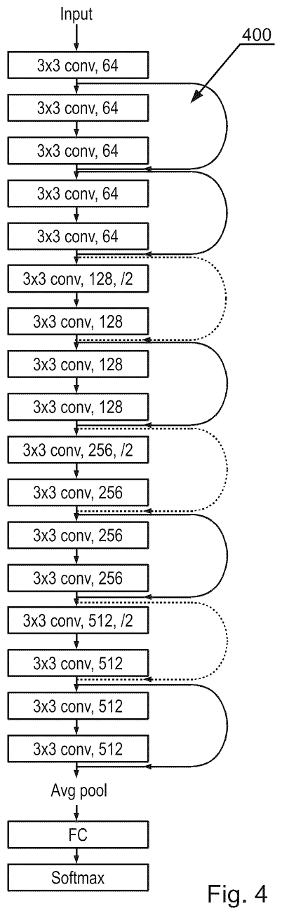

For example, for the classifying CNN 400 a convolutional neural network trained to perform typical classification task on a dataset such as ImageNet can be used, as shown in FIG. 4. The network is stripped of the top classification layers, so that the output of the network is a n-dimensional vector of the so-called bottleneck features. For example, for the ResNet18 architecture, the cutting point is denoted using a thick vertical line in the image below.

The features (ambeddings) are then normalized and represent a point in a n-dimensional space (embedding space). The k digital twin search can then be performed by finding k nearest neighbors of such a point in the n-dimensional space, assuming that the image entries in the database have undergone the same feature extraction process and each one has its representation as a point in this n-dimensional space. The advantage of such a solution is that it does not require additional training. The search can be sped up using feature dimensionality techniques such as principal component analysis (PCA) or by using approximate nearest neighbor search in place of brute force nearest neighbor search. The process can be sped up even further by transforming the embeddings into binary hashes. There are multiple methods to achieve this goal, ranging from simple or adaptive thresholding to more sophisticated approaches. For example, it is possible to use an approach as described in an article “Embarrassingly Simple Binary Representation Learning” (by Yuming Shen, Jie Qin, Jiaxin Chen, Li Liu, Fan Zhu, Ziyi Shen, Proceedings of the IEEE/CVF International Conference on Computer Vision (ICCV), 2019).

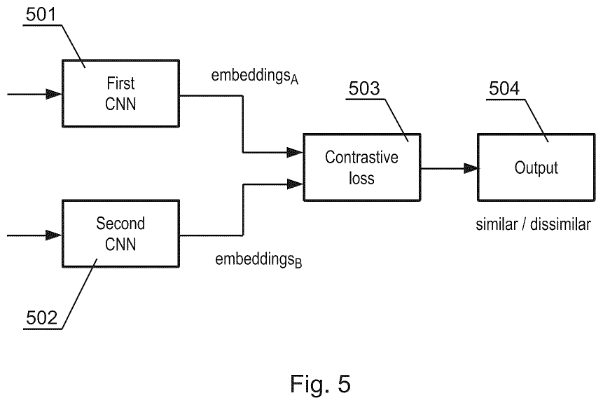

In case similarity and dissimilarity labels are available, they can be employed to train the neural network in a supervised regime, which in the most general case is using a siamese architecture and contrastive loss as shown in FIG. 5. The first and second convolutional neural networks 501, 502 in FIG. 5 share the same architecture and their weights are identical (they are essentially the same network). A variety of contrastive losses 503 can be used, such as contrastive loss, margin loss, triplet loss etc.

The networks 501, 502 may have an architecture as shown in FIG. 4—feature extractors typically used as the backend for image recognition with the network head removed to generate the embeddings. This so-called contrastive learning teaches the model to learn an embedding space in which similar examples are close while dissimilar ones are far apart, e.g., images belonging to the same class are pulled together, while distinct classes are pushed apart from each other. The trained network generates a point in the embedding space. With the training completed, the points in embedding space that are generated for similar images will be placed close in the embedding space, while the dissimilar images should generate points that are far apart.

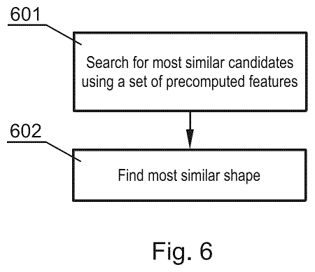

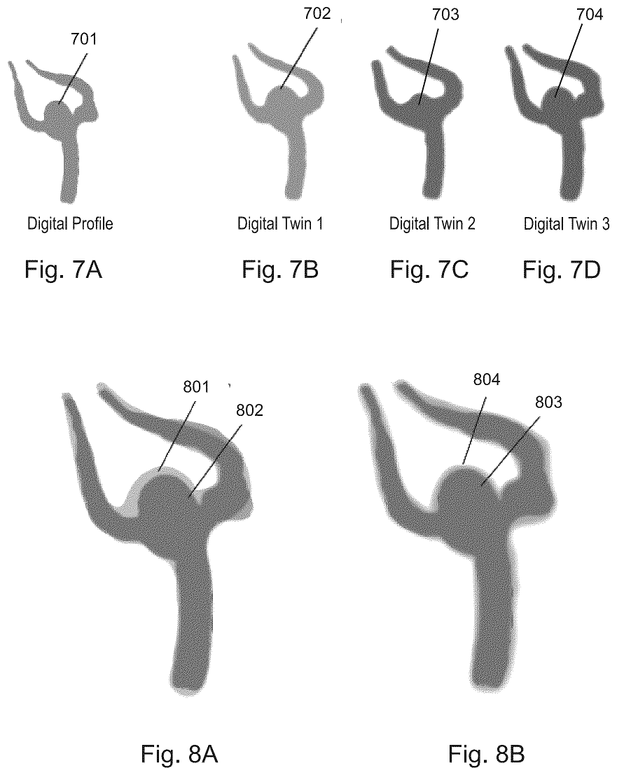

FIG. 6 shows a flowchart of a method for searching for a digital twin. The method uses a coarse-to-fine approach for best performance. First, in step 601, the most similar candidates may be selected from the patient history database 204 using a first technique, such real-valued or binary embeddings or other sets of precomputed features such as Hu moments or Zernike moments. Also, staple methods for description of binary shapes on 2D/3D images can be used. The resulting description is compact and invariant with respect to the rotation, translation, scaling etc. For example, when the search relates to a lesion, the search in step 601 can be performed using sets of moments calculated for the maximum intensity projections, or a set of moments calculated for the 3D shape of the lesion. The search involves only the computation of distance between the embeddings or shape descriptors, which is much faster than comparing the shapes directly, therefore it facilitates very fast database search. FIG. 7A shows an example of a digital profile 701 being examined and FIGS. 7B, 7C, 7D show profiles 702, 703, 704 of most similar candidates found in step 601. After the closest candidates are selected from the database, the most similar one is found in step 602 by using a second technique, such as 3D registration the query shape and the potential twin shapes and finding the one with minimum difference between the shapes. The difference can be computed using a dedicated metric, such as Hamming distance for binary vectors or Euclidean distance for real-valued descriptors. Other variables, categorical or numerical (such as age, sex) can also be used as a part of the compound database query.

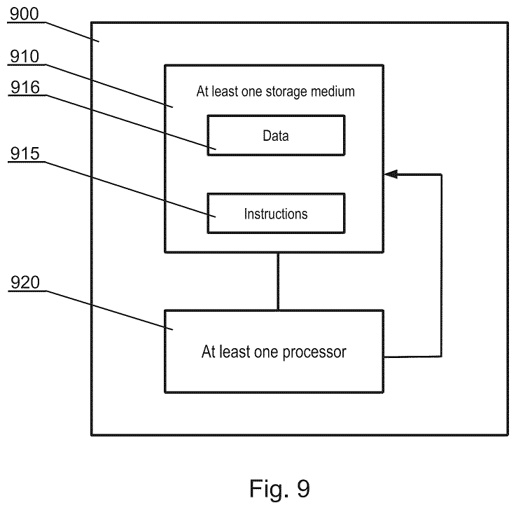

The functionality described herein can be implemented in a computer-implemented system 900, such as shown in FIG. 9. The system may include at least one non-transitory processor-readable storage medium that stores at least one of processor-executable instructions or data and at least one processor communicably coupled to the at least one non-transitory processor-readable storage medium. The at least one processor is configured to perform the steps of any of the methods presented herein. The computer-implemented system 900, for example a machine-learning system, may include at least one non-transitory processor-readable storage medium 910 that stores at least one of processor-executable instructions 915 or data 916; and at least one processor 920 communicably coupled to the at least one non-transitory processor-readable storage medium 910. The at least one processor 920 may be configured to (by executing the instructions 915) to perform the steps of any of the methods of FIG. 1 or 6.

Below are some examples of use of the invention.

### EXAMPLE 1

### 2D CT of the Chest—Search for a Similar 2D Image

A patient has lung nodules. A search for CTs in the database is performed that show nodules in similar densities and locations. 5 most similar images are found. For 3 of these images, it turns out that a drug was given that has better results than for the other 2 images. This can be a good indication of what treatment to use.

### EXAMPLE 2

### 3D Brain MRI—Search for a Similar 3D Volume

The scan revealed that the patient has a brain tumor. The system can find some of the best matching studies stored in the database and their associated treatment history. That way it can be determined whether radiation, chemotherapy, or perhaps surgical intervention is better to go straight to. A pool of retrieved similar historical cases with the full course of disease and treatment and their outcome is available in the database.

### EXAMPLE 3

### 3D Brain MRI—Search for Similar 2D or 3D Region of Interest

The scanned patient has an aneurysm. A search for patients with a similar aneurysm does not make sense to formulate as a search for an entire similar volume—it is better to cut out some region of interest containing its surroundings as the search query. The search can be performed in 2D or 3D—with either 3D region of interest volume data or its maximum intensity projection 2D image. With similar cases found, one has more data and information to suggest an effective treatment.

### EXAMPLE 4

### Fundus Images—Search for Similar 2D Eye Rear Images

The scanned patient exhibits symptoms of retinopathy, which include macular edema and microaneurysms. This imaging method produces 2D color images, based on which the most similar ones stored in the database can be found. The similarity is considered here as similar location and extent of the pathological changes. Based on the outcome of similar cases, the doctors can make an informed choice when selecting the preferred treatment (laser treatment, eye injections or eye surgery) or combination of treatments.

### EXAMPLE 5

### 3D MRI Scans of the Prostate—Search for Similar 3D Volumes

This imaging procedure is used mostly for prostate cancer diagnostics but will also reveal other conditions such as prostate infection or enlargement. Finding similar volumes (e.g., in terms of lesion placement, shape and size) among the stored cases and investigating their associated outcomes enables one to select the preferred course of action when it comes to biopsy or treatment: surgical procedure, cryotherapy, radiation therapy or chemotherapy.

### EXAMPLE 6

### 2D or 3D Ultrasound Image of the Liver—Similar 2D Image Search or 3D Volume Search

Certain liver diseases such as hepatitis, cirrhosis, and fatty liver (steatosis) can be reviewed in great detail from the ultrasound, as can pathological changes and lesions such as malignant tumors. Finding similar cases in the database can directly support the diagnosis and the review of treatment outcomes enables making an informed choice when it comes to the treatment options, as similar cases (e.g., in terms of lesion placement, shape and size or the degree of hepatitis of cirrhosis) will most probably respond to treatment similarly.

Although the invention is presented in the drawings and the description and in relation to its embodiments, these embodiments do not restrict or limit the presented invention. It is therefore evident that changes, which come within the meaning and range of equivalency of the essence of the invention, may be made. The presented embodiments are therefore to be considered in all aspects as illustrative and not restrictive. According to the abovementioned, the scope of the invention is not restricted to the presented embodiments but is indicated by the appended claims.

1. A method comprising:
receiving a medical image of an examined patient, the medical image covering an area or volume of the examined patient's anatomy;
inputting the medical image to a classifying neural network to generate descriptors;
receiving additional data of the examined patient;
providing an other patients history database comprising other patients' records, the records including the descriptors, the additional data and a clinical outcome of individual patients;
determining a patient from the other patient's history database being a closest match to the examined patients in terms of features of the descriptors to be a digital twin patient; and
presenting the clinical outcome of the digital twin patient.

2. The method according to claim 1, wherein the classifying neural network is an ImageNet.

3. The method according to claim 1, comprising determining the digital twin patient by finding a set of most similar candidates from the patient history database using a first technique and next finding the digital twin patient from the set of the most similar candidates using a second technique.

4. A computer-implemented system, comprising at least one nontransitory processor-readable storage medium that stores at least one of processor-executable instructions or data; and at least one processor communicably coupled to at least one nontransitory processor readable storage medium, wherein at least one processor is configured to perform the steps of the method according to claim 1.
